(19) United States
(12) Patent Application Publication
(10) Pub. No.: US 2019/0143136 A1
(43) Pub. Date: May 16, 2019

(54) METHOD AND SYSTEM FOR ENHANCED TREATMENT OF NEUROPSYCHIATRIC DISORDERS

(71) Applicant: THE REGENTS OF THE UNIVERSITY OF CALIFORNIA, Oakland, CA (US)

(72) Inventors: Aimee HUNTER, Santa Monica, CA (US); Andrew LEUCHTER, Los Angeles, CA (US)

(21) Appl. No.: 16/300,261

(22) PCT Filed: May 12, 2017
(86) PCT No.: PCT/US17/32314

§ 371 Date: Nov. 9, 2018

Related U.S. Application Data
(60) Provisional application No. 62/335,448, filed on May 12, 2016.
(60) Provisional application No. 62/468,168, filed on Mar. 7, 2017.

(51) Int. Cl. A61N 2/00; A61B 5/04; A61B 5/16; A61B 5/00; A61N 1/04; A61N 1/36

(52) U.S. Cl. CPC A61N 2/006; A61B 5/04017; A61B 5/165; A61N 1/36096; A61N 1/04; A61N 1/36025; A61B 5/4836

(57) ABSTRACT

The present invention provides systems and methods for enhanced treatment of depression and other neuropsychiatric disorders. The present invention personalizes neuromodulation treatment parameters based on their acute neurophysiological and behavioral effects in an individual patient to enhance clinical response to a course of repetitive Transcranial Magnetic Stimulation (rTMS) treatment and hasten time to recovery. The present invention assesses a range of stimulation parameters for their ability to produce specific acute effects on neurophysiologic function (specific pattern of change in resting-state EEG; current source density), and/or specific acute effects on the performance of specific cognitive tasks (increased reward sensitivity; diminished risk aversion; restored response bias deficiency) in an individual patient.

### CROSS-REFERENCE TO RELATED APPLICATIONS

This application claims priority to U.S. Provisional Patent Application No. 62/335,448, filed May 12, 2016, and to U.S. Provisional Patent Application No. 62/468,168, filed Mar. 7, 2017, the contents of which are each incorporated by reference herein in their entirety.

### BACKGROUND OF THE INVENTION

Depression is the second leading cause of medical disability in the United States and affects tens of millions of people each year. Neuromodulation therapies such as deep brain stimulation (DBS) and repetitive transcranial magnetic stimulation (rTMS) offer the potential to relieve symptoms in patients who are unresponsive to medication. Neuromodulation therapies are coming into widespread use. The effectiveness of these treatments varies widely, however, and may reflect in part the effectiveness of specific parameter settings (i.e., location, duration, frequency, pattern of stimulation) for a given patient. At present, there is a lack of understanding regarding how to adjust these parameters in order to achieve therapeutic benefit. Current clinical practice entails a protracted trial-and-error approach that begins with the use of a “standard” set of parameters and progresses through a sequence of alternative “standard” settings, each for weeks at a time, until determining a configuration that offers clinical benefit. Whereas many patients are helped using this strategy, a substantial proportion do not appear to show significant benefit from the initial trials of parameter settings, at which point the treatment modality may be abandoned altogether for lack of resources to continue, especially in the face of uncertain outcome.

There is a need in the art for an improved system and method for treatment of depression and other neuropsychiatric disorders. The present invention meets this need.

### SUMMARY OF THE INVENTION

In one aspect, the present invention relates to a method of treating neuropsychiatric disorders, comprising the steps of: assessing pretreatment severity of neuropsychiatric disorder symptoms using at least one evaluation test or interview; measuring pretreatment brain physiology and calculating at least one of pretreatment resting state delta and pretreatment resting state theta cordance using quantitative electroencephalography (qEEG); administering a series of repetitive Transcranial Magnetic Stimulation (rTMS) treatments to a target brain region; assessing the severity of neuropsychiatric disorder symptoms after a TMS treatment; measuring brain physiology and calculating at least one of posttreatment resting state delta and posttreatment resting state theta cordance using qEEG after a TMS treatment; and adjusting parameters for subsequent rTMS treatments to decrease at least one of resting state delta and resting state theta cordance below pretreatment resting state delta and pretreatment resting state theta cordance.

In one embodiment, the neuropsychiatric disorder is depression. In one embodiment, the resting state delta cordance is measured between 0.5 and 4 Hz. In one embodiment, the resting state theta cordance is measured between 4 and 8 Hz. 5. In one embodiment, the resting state theta cordance is measured in the central brain region. In one embodiment, the at least one evaluation test or interview is selected from the group consisting of: patient health questionnaire (PHQ-9), mini mental status exam (MMSE), mini international neuropsychiatric interview (MINI), the Montgomery-Asberg depression rating scale (MADRS), the Hamilton depression scale (HAM-D), the inventory of depressive symptomatology (IDS), clinical global impression improvement (CGI-I), clinical global impression severity (CGI-S), quality of life instrument (QOLI), and the Columbia suicide severity scale (C-SSRS). In one embodiment, qEEG further calculates alpha cordance, beta cordance, or both.

In one embodiment, the rTMS is administered to the left dorsolateral prefrontal cortex (left DLPFC). In one embodiment, the rTMS is administered at a baseline of 3000 pulses at 10 Hz. In one embodiment, the rTMS is administered in a continuous sequence alternating between stimulation and rest. In one embodiment, the stimulation time is 4 seconds. In one embodiment, the rest time is 26 seconds.

In one embodiment, the adjusted parameter is the location of the target brain location. In one embodiment, the adjusted parameter is the rTMS magnetic frequency. In one embodiment, the parameters are adjusted to decrease symptom severity as measured by HAM-D17 to a score equal to or below 7. In one embodiment, the parameters are adjusted to decrease symptom severity as measured by MADRS to a score equal to or below 12. In one embodiment, the parameters are adjusted to decrease symptom severity as measured by IDS-SR30 to a score equal to or below 12.

In another aspect, the present invention relates to a system for treating neuropsychiatric disorders, comprising: symptom severity evaluation software; an EEG device; a transcranial magnetic stimulation (TMS) device; and a computer platform.

In one embodiment, the symptom severity evaluation software is capable of administering at least one of the group consisting of: patient health questionnaire (PHQ-9), mini mental status exam (MMSE), mini international neuropsychiatric interview (MINI), antidepressant treatment history form (ATHF), the Montgomery-Asberg depression rating scale (MADRS), the Hamilton depression scale (HAM-D), the inventory of depressive symptomatology (IDS), clinical global impression improvement (CGI-I), clinical global impression severity (CGI-S), quality of life instrument (QOLI), and the Columbia suicide severity scale (C-SSRS).

In one embodiment, the EEG device is capable of measuring and recording neural oscillations in the frequency range of 0.1 Hz to 20 Hz. In one embodiment, the TMS device is programmable to control stimulation frequency, stimulation pattern, stimulation duration, repetition of stimulation, and stimulation intensity.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a flowchart depicting an exemplary method of treating neuropsychiatric disorders using repetitive Transcranial Magnetic Stimulation (rTMS).

FIG. 2 is a diagram of an exemplary system for treating neuropsychiatric disorders using rTMS.

FIG. 3A through FIG. 3D depict graphs for four subjects (Patient 1 though Patient 4, respectively) showing changes in clinical symptom severity (self-rated inventory of depressive symptomatology (IDS-SR), upper panel; patient health questionnaire (PHQ-9), lower panel), EEG time points, TMS treatment sessions, and changes in TMS treatment parameters, over the course of acute treatment. The x-axis reflects the number of days since the initial treatment session with the first session at Day 0. Diamond markers indicate each treatment session (usually 5 days per week initially, followed by a tapering period). All courses of treatment begin with stimulation (10 Hz) of the left dorsolateral prefrontal cortex (left DLPFC) as this is the standard protocol. Any point of change in treatment parameters (e.g., add in 1 Hz stimulation of the right DLPFC) is indicated by a dot along the x-axis. Dots displayed above the axis line indicate the time points at which an EEG was recorded; e.g. a dot labeled ‘EEG, 14’ indicates an EEG assessment performed 14 days after the first treatment session. ‘EEG, 0’ indicates the pretreatment baseline EEG recorded prior to stimulation. Finally, the triangles indicate clinical assessment time points with the raw score and date displayed next to each. For both the IDS-SR and PHQ-9 outcome measures, higher scores indicate more severe symptoms.

FIG. 4A through FIG. 4I depict topographic brain map series for four subjects showing EEG cordance measures at specified baseline, and change-from-baseline, time points. (Patient 1—FIG. 4A, FIG. 4B; Patient 2—FIG. 4C; Patient 3—FIG. 4D, FIG. 4E, FIG. 4F; Patient 4—FIG. 4G, FIG. 4H, FIG. 4I) EEGs are numbered sequentially from T1. . . . Tn where T1 is the pretreatment baseline EEG. Brain map series' showing the pretreatment EEG baseline (T1), and changes from T1, are generated for each patient. In cases where a later baseline is of interest, e.g., just prior to a change in treatment parameters, additional brain map series' are generated using a new baseline to examine acute changes in the EEG. For example, a map series using T6 as the baseline will show changes as compared to the 6th EEG. The first column shows baseline values, and subsequent columns show changes from that baseline. Increases are red; decreases are blue. EEG cordance measures in delta, theta, alpha and beta frequencies are shown in rows as labeled.

FIG. 5 is a diagram of an electrode montage for recording EEG data.

FIG. 6 is a graph showing the receiver operating characteristic (ROC) curve examining change in central cordance at one week as a predictor of improvement vs. non-improvement status after six weeks of rTMS was significant (p=0.001) with 0.97 area under the curve.

FIG. 7A through FIG. 7C depict scatterplots showing the change in central cordance at week 1 as a predictor of self-reported change in symptom severity (IDS-SR) at week 2 (FIG. 7A), week 4 (FIG. 7B), and week 6 (FIG. 7C).

FIG. 8A through FIG. 8C depict topographic maps of theta cordance in clinical rTMS participants after 1 week of rTMS treatment. FIG. 8A is the week 1 topographic map of participants who were improvers (n=12) after 6 weeks of rTMS treatment. FIG. 8B is the week 1 topographic map of participants who were non-improvers (n=6) after 6 weeks of rTMS treatment. Increases are red; decreases are blue. FIG. 8C is the topographic map of the statistical significance of differences in theta cordance between CGI-I improvers vs. non-improvers. Electrode recording sites colored in yellow or white have the lowest t-test p-values, indicating the greatest probability of statistical significance.

### DETAILED DESCRIPTION

The present invention provides systems and methods for enhanced treatment of depression and other neuropsychiatric disorders. The present invention personalizes neuromodulation treatment parameters based on their acute neurophysiological and behavioral effects in an individual patient to enhance clinical response to a course of repetitive Transcranial Magnetic Stimulation (rTMS) treatment and hasten time to recovery. The present invention assesses a range of stimulation parameters for their ability to produce specific acute effects on neurophysiologic function (specific pattern of change in resting-state EEG; current source density), and/or specific acute effects on the performance of specific cognitive tasks (increased reward sensitivity; diminished risk aversion; restored response bias deficiency) in an individual patient.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

Method of Treating Neuropsychiatric Disorders

The present invention is in part based upon measurement of electroencephalographic (EEG) power (or related measures that are derived from power) within the 0.5-20 Hz total frequency range in order to provide a personalized delivery of repetitive Transcranial Magnetic Stimulation (rTMS) to relieve symptoms of various neuropsychiatric disorders. In some embodiments, the neuropsychiatric disorder is Major Depressive Disorder (MDD). The invention integrates measurements of EEG absolute power (the intensity of energy measured in square microvolts) and relative power (the percentage of energy in a particular frequency band) in sub-bands within the total frequency range. Changes commonly are seen in multiple frequency bands within the total range including the delta (0.5-4 Hz), theta (4-8 Hz), and beta (12-20 Hz) ranges.

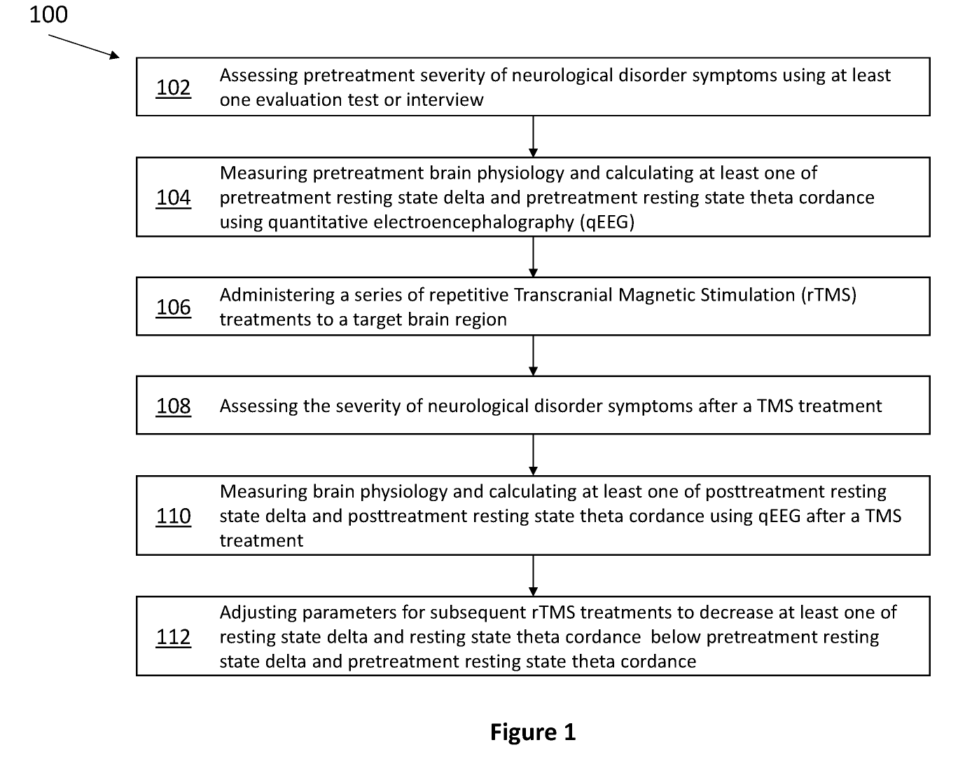

Referring now to FIG. 1, an exemplary method 100 of administering rTMS is presented. Method 100 begins with step 102, wherein pretreatment severity of neuropsychiatric disorder symptoms are assessed using at least one evaluation test or interview. In step 104, pretreatment brain physiology is measured, and at least one of pretreatment resting state delta and pretreatment resting state theta cordance is calculated using quantitative electroencephalography (qEEG). In step 106, a series of repetitive Transcranial Magnetic Stimulation (rTMS) treatments are administered to a target brain region. In step 108, the severity of neuropsychiatric disorder symptoms are assessed after a TMS treatment. In step 110, brain physiology is measured and at least one of posttreatment resting state delta and posttreatment resting state theta cordance is calculated using qEEG after a TMS treatment. In step 112, the parameters for subsequent rTMS treatments are adjusted to decrease at least one of resting state delta and resting state theta cordance below pretreatment resting state delta and pretreatment resting state theta cordance.

The present invention in part monitors changes in treatment efficacy as indicated by evaluation tests or interviews commonly used by persons skilled in the art. Non-limiting examples of evaluation tests or interviews include: patient health questionnaire (PHQ-9), mini mental status exam (MMSE), mini international neuropsychiatric interview (MINI), antidepressant treatment history form (ATHF), the Montgomery-Asberg depression rating scale (MADRS), the Hamilton depression scale (HAM-D), the inventory of depressive symptomatology (IDS), clinical global impression improvement (CGI-I), clinical global impression severity (CGI-S), quality of life instrument (QOLI), and the Columbia suicide severity scale (C-SSRS).

The present invention in part also monitors changes in EEG power measures between a recordings performed before the start of a course of rTMS treatment and at regular intervals after the start of a course of rTMS treatment, or before and after a change in the parameters used to deliver rTMS treatment. EEG measurements may be taken using any suitable EEG measuring device known in the art, such as a 21-channel wireless, dry-sensor headset. The EEG measurements are used to calculate at least delta and theta cordance using quantitative EEG (qEEG). In some embodiments, alpha and beta cordances are also calculated.

The series of rTMS treatments begin with a baseline administration of about 3000 pulses delivered at 10 Hz to the left dorsolateral prefrontal cortex (DLPFC). Additional initial parameters may include grouping the pulses in 30 second cycles, with a stimulation on-time of 4 seconds and an off-time of 26 seconds, for a total treatment time of about 37.5 minutes. rTMS treatments may be administered at regular intervals, such as once a day, once every two days, once every three days, etc. In some embodiments, rTMS treatments may be spaced irregularly as would be determined to be appropriate by an operator.

After certain rTMS treatments, the severity of neuropsychiatric disorder symptoms are re-assessed using evaluation tests or interviews and EEG is re-measured to calculate new resting state delta and resting state theta cordances. In some embodiments, the evaluation tests or interviews and EEG measurements occur at least 12 hours after an rTMS treatment to reduce the likelihood of lingering effects from rTMS treatment affecting the assessment and EEG measurement. The timing of the symptom assessment and EEG measurement can be after every one rTMS treatment, after every two rTMS treatments, after every three rTMS treatments, etc. In some embodiments, the timing may be reduced or increased based on the results of past evaluation tests or interviews and EEG measurements. For example, little to no change may warrant an increase in timing, while large changes may warrant a decrease in timing.

In some embodiments, the symptom severity assessments and EEG measurements after rTMS treatments indicate an effective decrease in severity of symptoms and a decrease in resting state delta and resting state theta cordance. An effective decrease in severity of symptoms may be indicated, for example, by a HAM-D17 score equal or below 7, a MADRS score equal to or below 12, or an IDS-SR30 score equal to or below 12. Subsequent rTMS treatments may be performed using the same parameters as baseline administration.

In some embodiments, the symptom severity assessments and EEG measurements after rTMS treatments indicate insufficient, no change, or even an increase in severity of symptoms, in resting state delta and resting state theta cordance, or both. An insufficient change in severity of symptoms may be indicated, for example, by a HAM-D17 score above 7, a MADRS score above 12, or an IDS-SR30 score above 12. In order to effectively decrease symptom severity and resting state cordance, subsequent rTMS treatments are administered with parameters adjusted from baseline. Such parameters include changing the location of the target brain region, the numbers or intensity of the magnetic pulses, or the pattern or frequency of the pulses. Example adjustments include increasing or decreasing stimulation frequency, such as in 1 Hz increments; supplementing left DLPFC stimulation with right DLPFC stimulation; stimulating the dorsomedial prefrontal cortex (DMPFC) instead of or in addition to the DLPFC; changing the sequence of target brain region stimulation; and changing the duration of target brain region stimulation. For example, in one embodiment, the high frequency (e.g., 10 Hz) pulses administered to the left DLPFC may be followed by a number of low frequency (e.g., 1 Hz) pulses administered to the right DLPFC within a single treatment session. In certain embodiments, the adjustments may include administering “theta burst stimulation” (TBS) patterns to the left DLPFC, the right DLPFC, or both. TBS represents a rapid conditioning method that can produce a powerful effect on cortical excitability in a short period of time (<10 minutes of stimulation). TBS is based upon long term depression (LTD) and long term potentiation (LTP) inducing stimulation patterns developed in animal models and involves a triad of high-frequency (such as 50 Hz) bursts, with an interburst interval of about 200 ms (such as 5 Hz). A specific pattern of TBS, i.e., ‘intermittent TBS’ (iTBS), has been shown to increase cortical excitability with effects lasting beyond the stimulation session, whereas ‘continuous TBS’ (cTBS) has been shown to decrease cortical excitability beyond the treatment session (Huang Y Z et al., “Theta burst stimulation of the human motor cortex.” Neuron 45.2 (2005): 201-206.).

In some embodiments wherein the rTMS treatment parameters are adjusted, the symptom severity assessments and EEG measurements are repeated to determine whether the adjusted parameters are effective in decreasing symptom severity and resting state delta and resting state theta cordance. If the adjustment is effective in decreasing delta and theta cordance and symptoms, then rTMS treatments may proceed with similar parameters. If the adjustment is ineffective, then the parameter adjustment steps are repeated.

System for Treating Neuropsychiatric Disorders

In another aspect, the present invention provides a system for treating neuropsychiatric disorders. The system provides the components and devices necessary for performing the steps of the methods presented elsewhere herein.

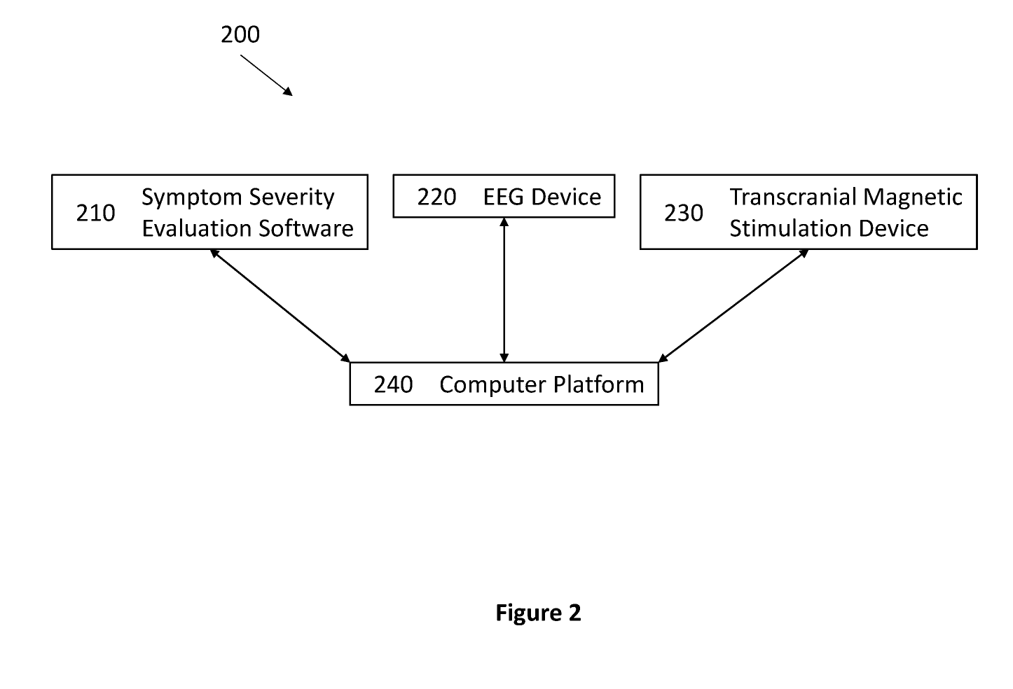

Referring now to FIG. 2, a diagram of an exemplary system 200 for treating neuropsychiatric disorders is depicted. System 200 comprises symptom severity evaluation software 210, EEG device 220, transcranial magnetic stimulation device 230, and computer platform 240.

In some embodiments, symptom severity evaluation software 210 is a software platform that delivers clinical questionnaires or conducts structured interviews for patients to report their symptoms, the severity of their symptoms, and to answer other questions relevant to the status of their neuropsychiatric disorder. The software can comprise one or more evaluation tests or interviews commonly used in the art, including: PHQ-9, MMSE, MINI, ATHF, MADRS, HAM-D, IDS, CGI-I, CGI-S, QOLI, and C-S SRS.

In some embodiments, EEG device 220 can be any suitable EEG device capable of measuring and recording neural oscillations in the frequency ranges of 0.1 Hz to 20 Hz. Preferably, the EEG device is capable of measuring and recording at least delta waves in the 0.5-4 Hz range and theta waves in the 4-8 Hz range.

In some embodiments, transcranial magnetic stimulation device 230 can be any suitable device capable of delivering magnetic stimulation to a desired region of the brain. The device is preferably programmable to control stimulation frequency, stimulation pattern, stimulation duration, repetition of stimulation, stimulation intensity, and the like.

As contemplated herein, computer platform 240 may comprise any computing device as would be understood by those skilled in the art, including desktop or mobile devices, laptops, desktops, tablets, smartphones or other wireless digital/cellular phones, televisions or other thin client devices as would be understood by those skilled in the art.

Computer platform 240 is fully capable of sending commands to the components of system 200 and interpreting received signals as described herein throughout. In certain embodiments, portions of the system may be computer operated, or in other embodiments, the entire system may be computer operated. The computer platform can be configured to control parameters such as stimulation frequency, stimulation pattern, stimulation duration, repetition of stimulation, stimulation intensity, and the like. The computer platform can be configured to record received signals, and subsequently interpret the received signals in real-time. For example, the computer platform can also be configured to record EEG measurements and to perform the necessary calculations to determine cordance. The computer platform may also be configured to interpret any received signals as images and subsequently transmit the images to a digital display. The computer platform may further provide a means to communicate the received signals and data outputs, such as by projecting one or more static and moving images on a screen, emitting one or more auditory signals, presenting one or more digital readouts, providing one or more light indicators, providing one or more tactile responses (such as vibrations), and the like. In some embodiments, the computer platform communicates received signals and data outputs in real-time, such that an operator may adjust the use of the device in response to the real-time communication.

In some embodiments, symptom severity evaluation software 210 may reside on computer platform 240. In other embodiments symptom severity evaluation software 210 resides on a separate platform. The software may also include standard reporting mechanisms, such as generating a printable results report, or an electronic results report that can be transmitted to any communicatively connected computing device, including computer platform 240. Likewise, particular results of the aforementioned system can trigger an alert signal, such as the generation of an alert email, text or phone call, to alert an operator of the particular results. Further embodiments of such mechanisms are described elsewhere herein or may be standard systems understood by those skilled in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out the preferred embodiments of the present invention, and are not to be construed as limiting in any way the remainder of the disclosure.

### Example 1: Determining Personalized Transcranial Magnetic Stimulation (TMS) Parameters to Enhance Clinical Treatment Outcomes in Major Depression and Other Neuropsychiatric Disorders

Evidence indicates that within moments of beginning repetitive TMS (rTMS) administration, the effects of stimulation spread throughout brain networks to change both neurophysiological function and behavior (e.g., performance on cognitive tasks). It is expected that real-time or proximal monitoring of the neurophysiologic (e.g., EEG) and behavioral effects of neuromodulation can be used to adjust the site and mode of delivery of stimulation to enhance therapeutic efficacy. The study described herein examines the association between early neurophysiologic effects of stimulation at various parameters, and subsequent therapeutic effectiveness with continued treatment at the given parameter settings.

A critical challenge in the management of major depressive disorder (MDD) is the selection of treatment for each individual patient. Although treatments with depression can restore people's lives, with any treatment modality there are some individuals who do not achieve complete remission of symptoms, whether the intervention is pharmacological, psychological, or somatic. While predictors of response for some treatment modalities have been proposed for groups of patients, the translation of these predictors to individualized patient care has remained elusive. Further, within any treatment modality there exist many variations or treatment parameters that are potentially important in determining and individual's response. A biomarker that could predict an individual's likelihood to respond to rTMS would be of great use to clinicians and patients in making decisions regarding this treatment modality. Further, a biomarker that could predict efficacy of specific rTMS parameters for the individual patient would have great value for personalizing rTMS treatment and optimizing outcomes.

EEG biomarkers have long held promise as predictors of antidepressant treatment outcome (Hunter, A M et al., Psychiatric Clinics of North America 30.1 (2007): 105-124). Earlier work introduced a new physiologic biomarker of treatment response that was initially developed in studies of selective serotonin reuptake inhibitor (SSRI) and mixed-action antidepressant medications (Cook, I A et al., Seminars in clinical neuropsychiatry. Vol. 6. No. 2. 2001; Cook, I A et al., Neuropsychopharmacology 27.1 (2002): 120-131; Cook, I A et al., Journal of psychiatric research 39.5 (2005): 461-466; Cook, I A et al., Psychiatry Research: Neuroimaging 174.2 (2009): 152-157; Leuchter, A F et al., American Journal of Psychiatry (2002)). The EEG-based “cordance” biomarker, measured within the first week of treatment, has repeatedly and independently been demonstrated to predict response to reuptake inhibitor antidepressant treatment at the end of eight weeks (Kopecek, M et al., 14th European Congress of Psychiatry, AEP. Nice. 2006; Bareš M et al., J Psychiatr Res. 41(3-4):319-25; Bareš, M et al., European Psychiatry 23.5 (2008): 350-355). Additionally, the magnitude of early physiologic change has been associated with the completeness of clinical response. Studies of other treatments for depression suggest that early changes in cordance may be linked to clinical response across modalities. Administration of the NMDA antagonist ketamine, a novel agent that has a rapid antidepressant effect, was shown to induce a decrease in prefrontal cordance similar to that seen in monoamineric-based antidepressants (Horacek, J et al., Psychological medicine 40.09 (2010): 1443-1451). Two recent studies have examined cordance as a predictor of rTMS outcome (Arns, M et al., Brain Stimulation 5.4 (2012): 569-576; Bares, M et al., Clinical EEG and neuroscience 46.2 (2015): 73-80). Whereas the Arns et al. (2012) study examined prefrontal cordance (delta and theta) only at the pretreatment baseline and did not address specificity for rTMS response, the Bares et al. (2015) study examined the treatment-emergent prefrontal theta cordance biomarker as a predictor of outcome to a specified treatment. (Leuchter, A F et al., Current psychiatry reports 12.6 (2010): 553-562). Utilizing this paradigm, a recent study demonstrated that decreases in prefrontal cordance after one week of right prefrontal slow (1 Hz) rTMS predicted clinical response after four weeks of treatment (Bares, M et al., Clinical EEG and neuroscience 46.2 (2015): 73-80).

The present study utilizes the EEG cordance measure, a measure that integrates absolute and relative power. The cases demonstrate that rTMS treatment that is going to lead to remission of depressive symptoms causes a reduction in cordance in the theta and delta frequency bands several weeks prior to the onset of clinical benefit. A decrease in cordance in the theta frequency band alone may be associated with clinical improvement, but not with a complete remission of depressive symptoms. Following a change in parameters, a decrease in cordance in the theta frequency band was accompanied by a decrease in the delta frequency band as well, which preceded a complete remission of depressive symptoms. Four individual patients are presented, each having four distinct and clinically-relevant trajectories of symptom change.

Patient 1: Responder, Linear Clinical Improvement, No Change in Treatment Parameters

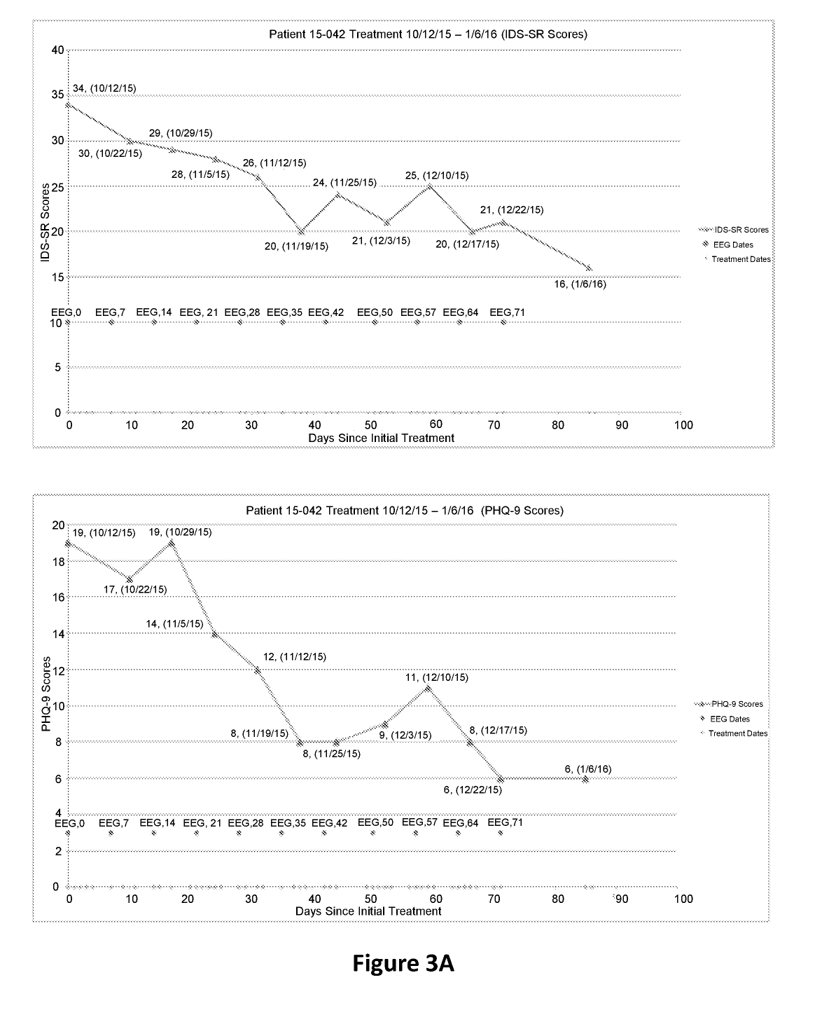

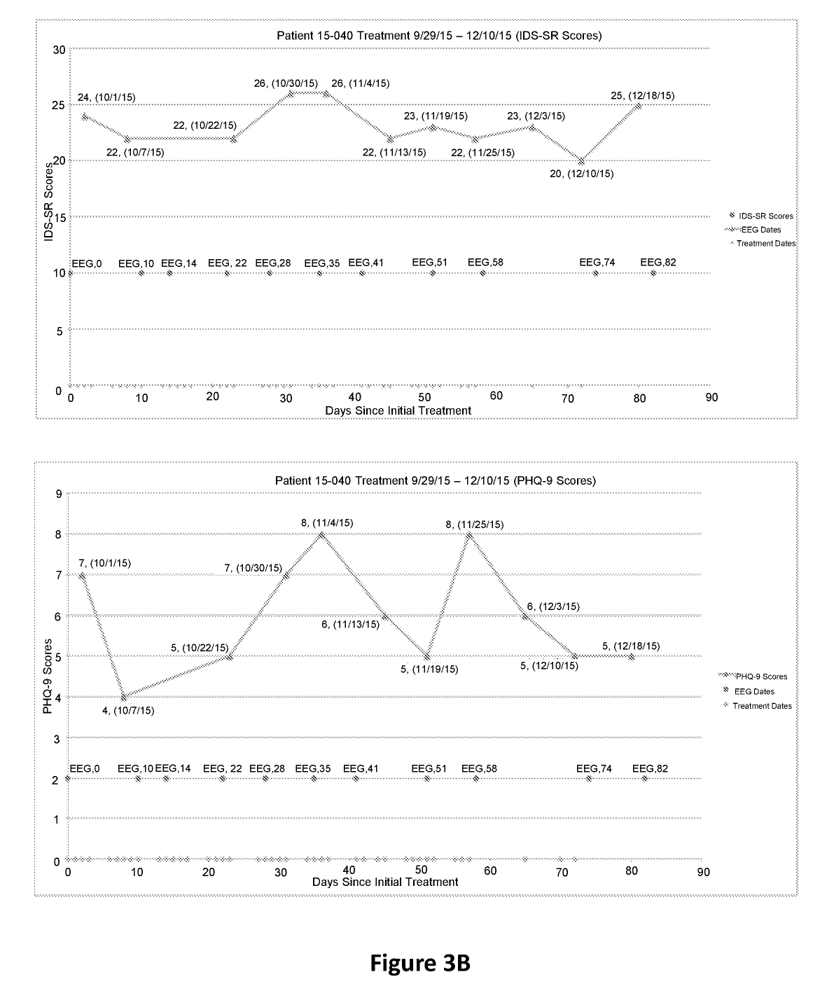

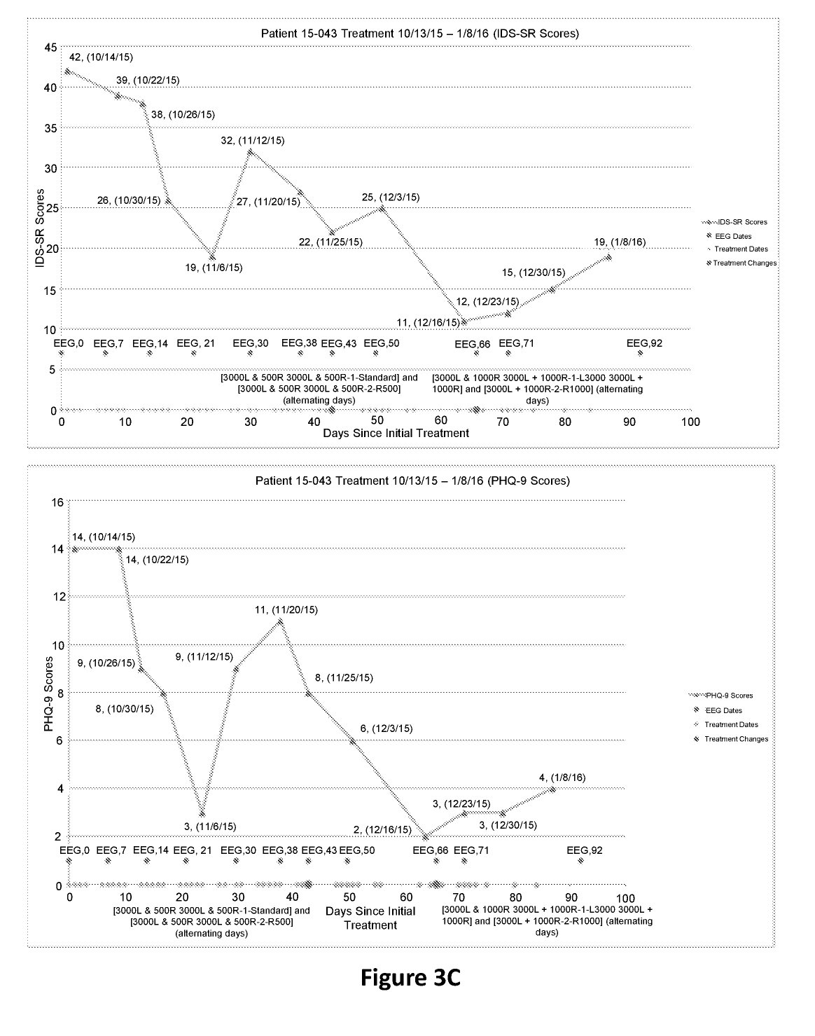

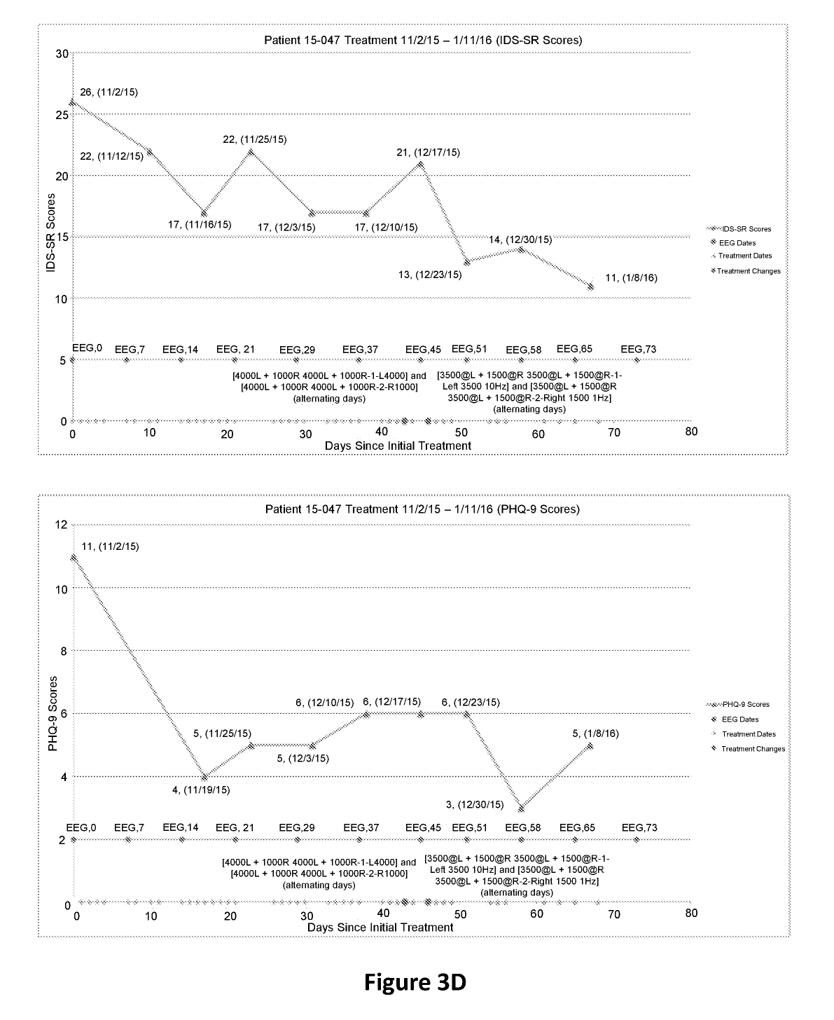

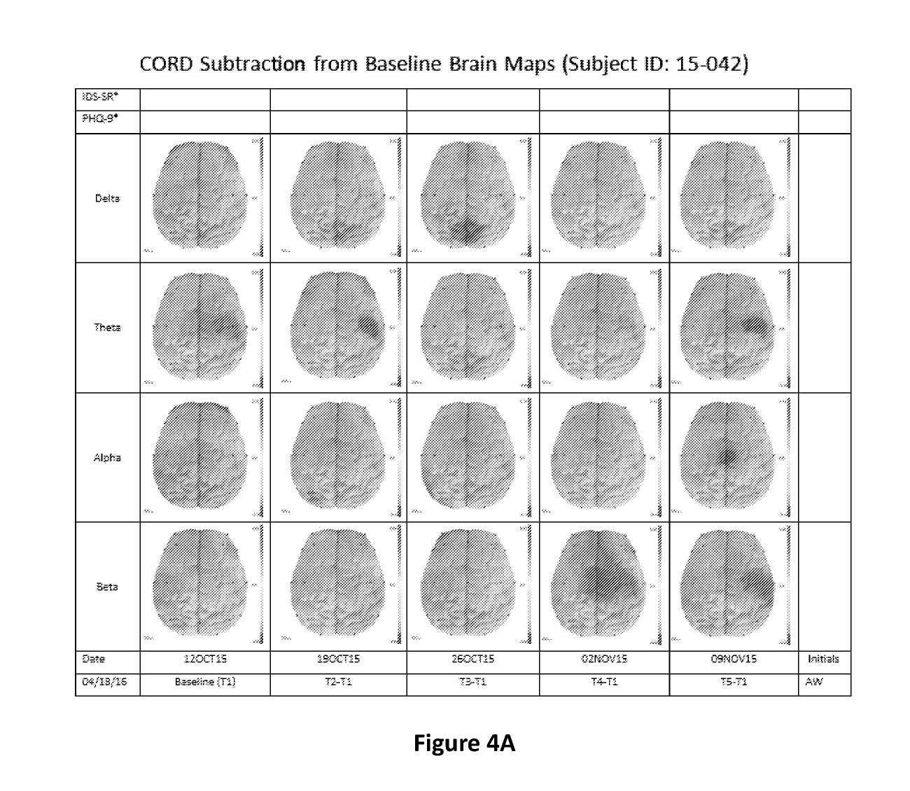

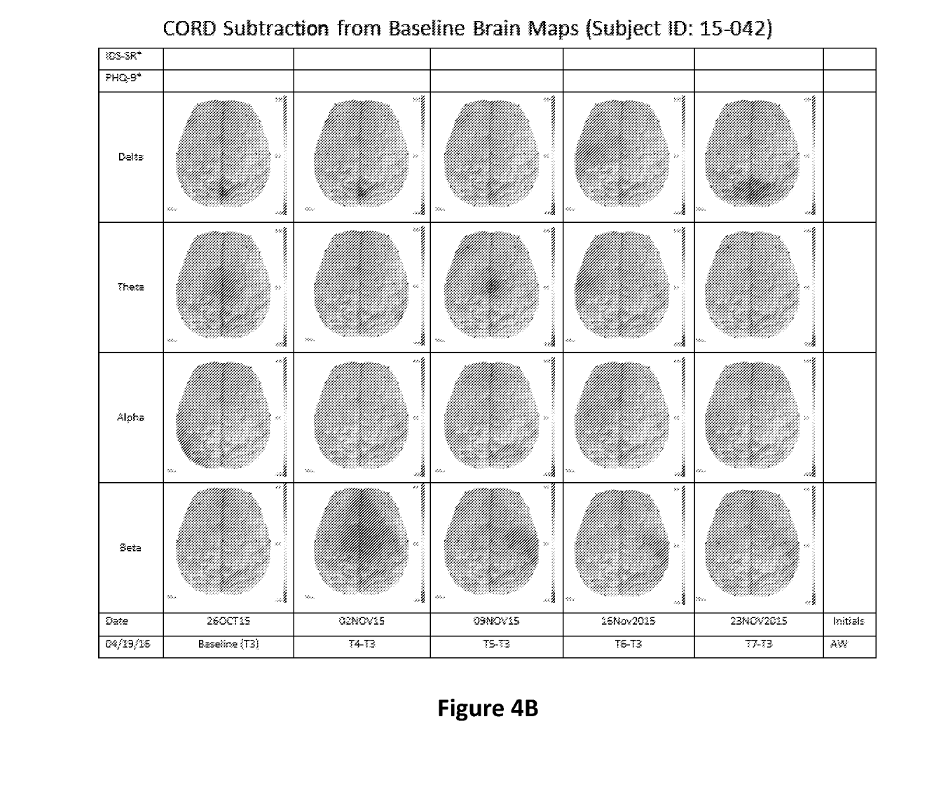

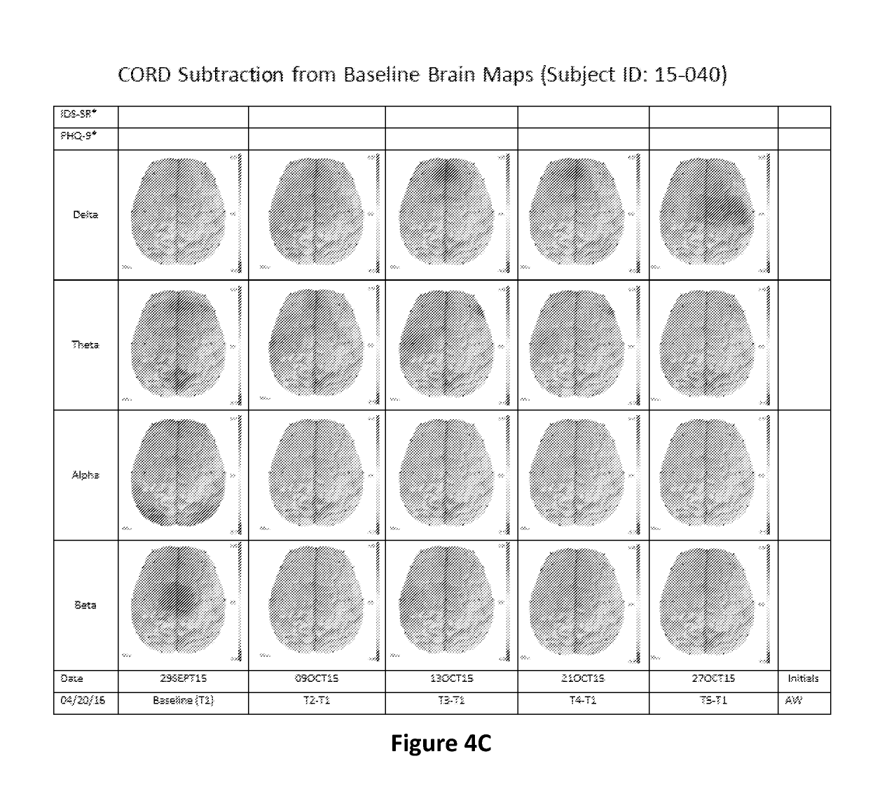

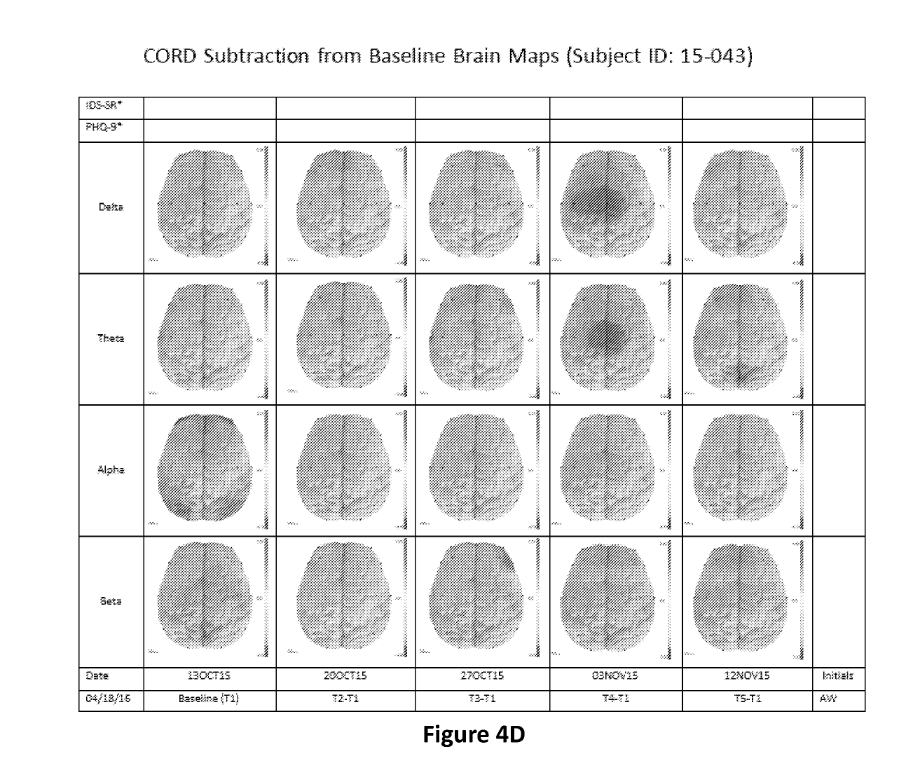

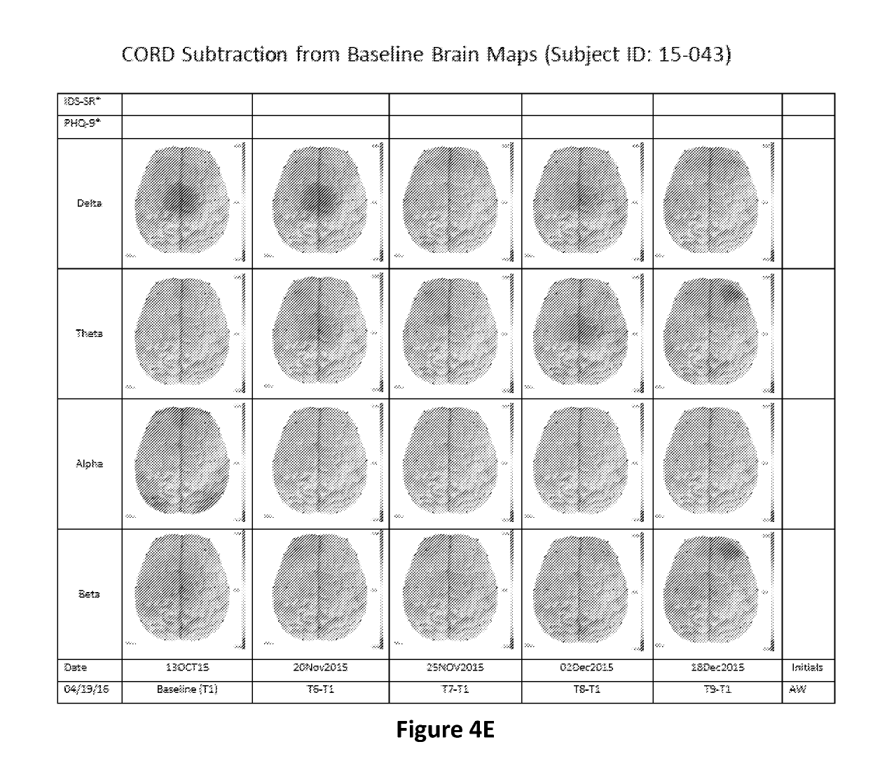

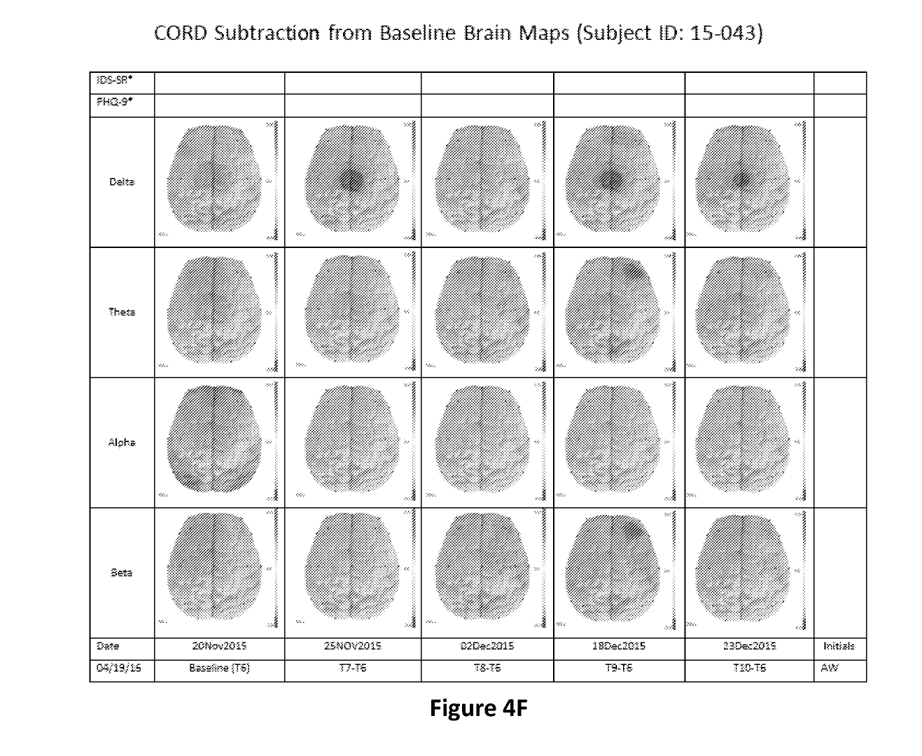

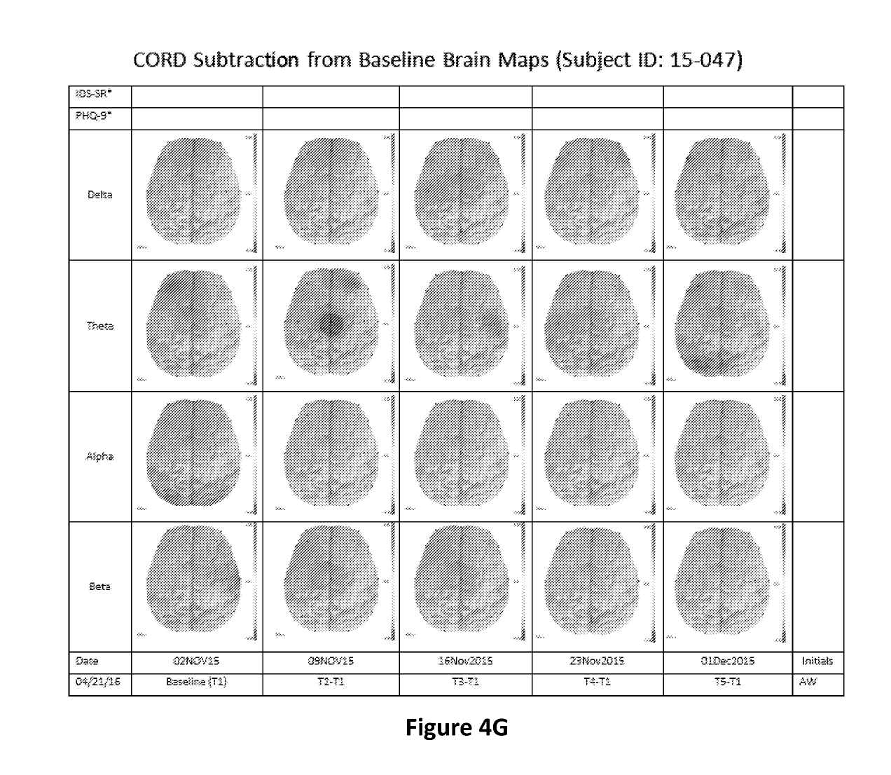

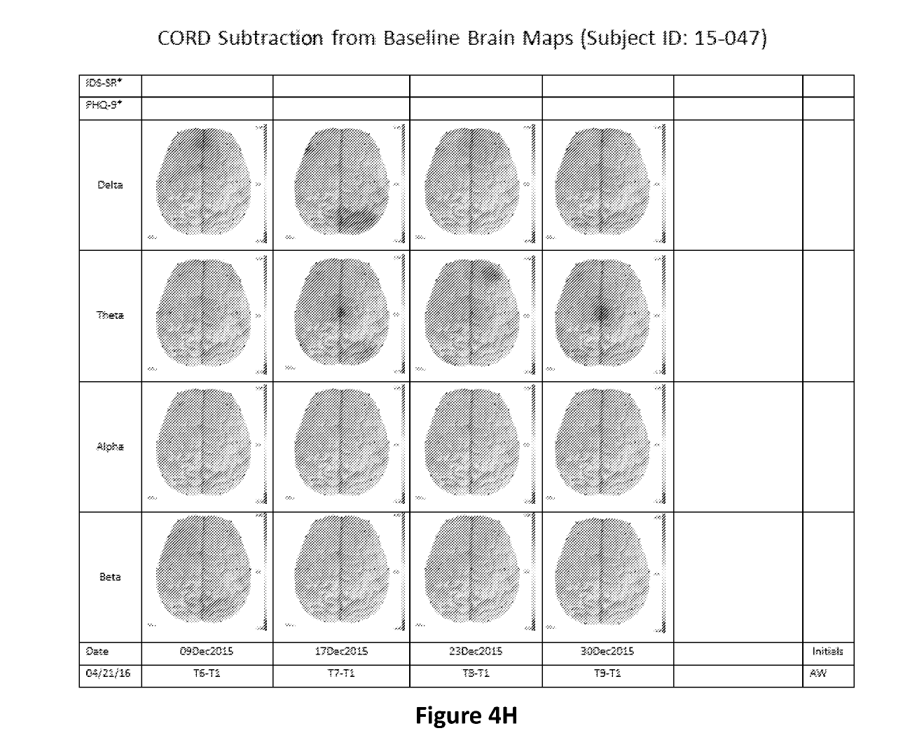

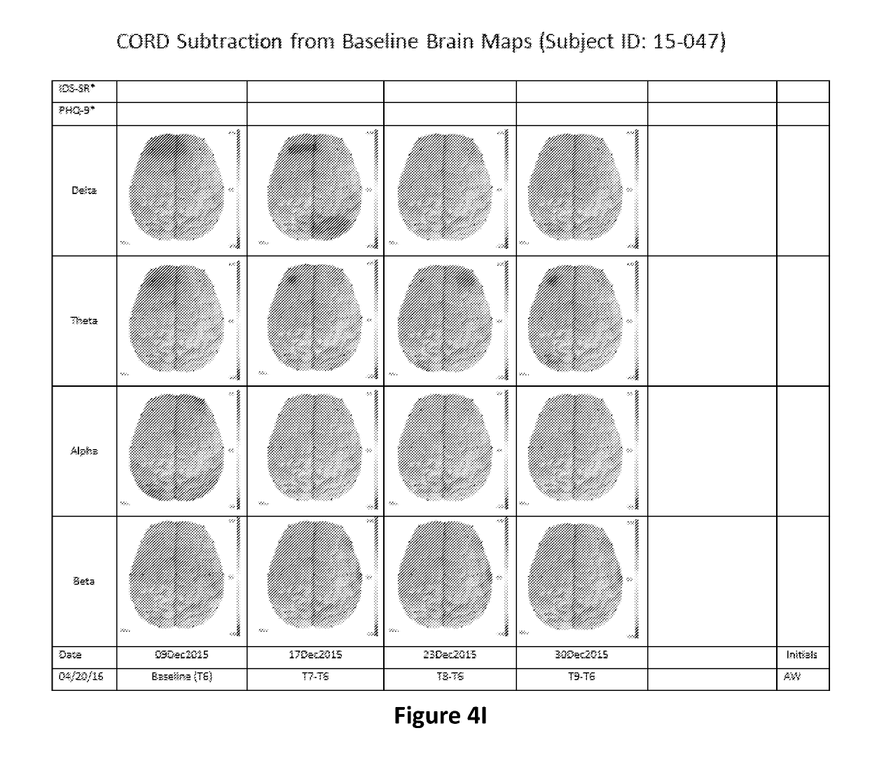

Patient 1 showed significant sustained improvement over the course of TMS treatment without requiring any change in parameters from the initial 10 Hz stimulation of left DLPFC. Symptom severity decreased steadily culminating in a reduction of 53% on the self-rated inventory of depressive symptomatology (IDS-SR), and 68% on the patient health questionnaire (PHQ-9) at Day 85 (FIG. 3A). Brain maps show EEG changes (decreases) in theta cordance in right central regions from T1 baseline, observed beginning with the first post-treatment EEG (7 days after beginning treatment) (FIG. 4A). Decreases in theta cordance from T1 are maintained over the first five EEGs (Day 28) (FIG. 4A). Shifting the baseline to T3, resulted in a similar pattern of change (i.e. further decreases in theta cordance) suggesting stability of this measure as an indicator of continued clinical improvement with ongoing treatment using standard parameters (FIG. 4B).

Patient 2: Non-Responder, Narrow Range of Symptom Fluctuation without Discernable Pattern of Improvement (No Change in Treatment Parameters)

Patient 2 showed no significant change in symptoms at any point over the course of treatment. IDS-SR scores never deviated more than 2 points from the baseline value, and PHQ-9 scores never deviated more than 3 points from baseline (FIG. 3B). Brain maps do not show the prominent central, or right central decreases in theta cordance that were observed in Patient 1's (responder) maps (FIG. 4C). Although there is some decrease in theta cordance, the topography and time course differ from Patient 1 (responder); Patient 2 shows a transient decrease in theta cordance posteriorly, and only a small focal decrease in theta cordance in the right frontal region (FIG. 4C). Unlike Patient 1 (responder), these decreases in theta cordance are limited and are not accompanied by an increase in fast-wave (i.e. beta) activity (FIG. 4C). If anything, there appears to be a decrease in beta and an increase in slow-wave (delta cordance) over time (FIG. 4C).

Patient 3: Initial Nonsustained Clinical Response, Subsequent Sustained Clinical Response Following Change in Treatment Parameters

Patient 3 showed an early, dramatic, but nonsustained reduction in symptoms, which is common in clinical practice or treatment trials for depression. The pattern is often thought to reflect a ‘placebo response,’ a frequently transient clinical response to non-specific factors associated with the treatment setting (rather than response to the specific intervention). Patient 3 showed reductions of 55% and 79% on the IDS-SR and PHQ-9, respectively in the first four weeks of treatment, and symptoms rebounded between weeks 4-6 despite continued treatment with the initial TMS parameters (FIG. 3C). Return of symptoms prompted the treating physician to alter treatment going forward to include right-sided slow (1 Hz) stimulation of DLPFC; this change in parameters marked the onset of relatively sustained improvement on the PHQ-9 (63% initial reduction until taper of TMS treatment; 50% reduction at end of tapering), and the IDS-SR (36% reduction until taper; 27% reduction at end of taper) (FIG. 3C). Interestingly, brain maps showing changes from T1 baseline did not indicate any notable decreases in theta (or delta) cordance during treatment with the initial parameters; there was no indication of a decrease in slow-wave (i.e., delta or theta) cordance during the first phase of treatment (FIG. 4D, FIG. 4E)). The data demonstrate that a decrease in slow-wave power-related measures (theta/delta cordance or similar) might be an indicator of specific response to TMS rather than ‘placebo’ response. Examination of cordance changes from T6 (the time point immediately prior to change in treatment parameters) revealed immediate, prominent, and relatively sustained decreases in delta and/or theta cordance following the change in treatment settings that appeared to result in more durable clinical improvement (FIG. 4F).

Patient 4: Initial Partial Responder, Further Clinical Response Followed Change in Treatment Parameters

Patient 4 shows what may be considered a “staged” pattern of clinical response where the patient exhibits partial improvement with an initial treatment intervention but requires additional, or adjusted treatment to achieve full response. Based on IDS-SR scores, symptom improvement for this patient appeared to plateau at about 38 days from the initial session. Whereas Patient 4 showed reductions of 35% and 45%, respectively, on the IDS-SR and PHQ-9 while receiving standard treatment, the IDR-SR score was still at 17, a level that is above the threshold for remission (FIG. 3D). Treatment parameters were adjusted beginning at Day 44, and within a week the IDS-SR score moved to 13, and finally dropped to 11 at Day 66 (last assessment time point) (FIG. 3D).

In this case, brain maps show an initial large central decrease in theta cordance after the first week of treatment that appears somewhat less pronounced until the change in parameters following the 6th EEG at which point the large central decreases in theta cordance are evidenced consistently though the 9th EEG (FIG. 4G, FIG. 4H). Changes from T6 baseline, following the change in treatment parameters, show further decreases in theta and delta cordance (FIG. 4I).

### Example 2: Change in Quantitative EEG Theta Cordance as a Predictor of Repetitive Transcranial Magnetic Stimulation (rTMS) Clinical Outcome in Major Depressive Disorder

Repetitive Transcranial Magnetic Stimulation (rTMS) has proven efficacy for treatment of Major Depressive Disorder (MDD) in patients who have not responded fully to a course of treatment with antidepressant medication (George M S et al., Biological psychiatry 48.10 (2000): 962-970; George M S et al., Archives of general psychiatry 67.5 (2010): 507-516; Fitzgerald P B et al., Archives of General Psychiatry 60.10 (2003): 1002-1008; Avery D H et al., Biological psychiatry 59.2 (2006): 187-194; O'Reardon J P et al., Biological psychiatry 62.11 (2007): 1208-1216). However, as with other treatments for MDD, there is large inter-individual variability in outcomes (Brunelin J et al., L′Encephale 33.2 (2006): 126-134; Chung S W et al., Brain stimulation 8.6 (2015): 1010-1020), whereas those who do benefit may not show clinical improvement until after receiving several weeks of a 6-week acute course of rTMS. Because of the cost and logistical challenge of receiving treatment five days per week, a biomarker that could predict an individual's likelihood to respond would be of great use in making decisions regarding whether to start or continue rTMS treatment and to guide adjustment of treatment parameters to increase treatment effectiveness.

Quantitative electroencephalography (qEEG) biomarkers have long shown promise as leading indicators of outcome to other treatments for depression (Cook I A et al., Neuropsychopharmacology 27.1 (2002): 120-131; Hunter A M et al., Psychiatric Clinics of North America 30.1 (2007): 105-124; Leuchter A F et al., Annals of the New York Academy of Sciences 1344.1 (2015): 78-91; Leuchter A F et al., Journal of Psychiatric Research 84 (2017): 174-183), but have not been examined extensively in the context of clinical rTMS. There is some evidence of baseline neurophysiologic (EEG and ERP) predictors of non-response to rTMS; however these largely overlap with predictors of non-response to antidepressant medication (Arns M et al., Brain stimulation 5.4 (2012): 569-576) and may suggest the presence of treatment-resistant depression rather than the likelihood of improvement with rTMS specifically.

Treatment-emergent EEG biomarkers have been posited to constitute “response endophenotypes” (Leuchter A F et al., Dialogues Clin Neurosci 11.4 (2009): 435-446) or “intermediate phenotypes” (Leuchter A F et al., Annals of the New York Academy of Sciences 1344.1 (2015): 78-91) that may offer greater specificity than pretreatment markers for predicting response to a given treatment, thus having greater utility for personalized medicine decision-making (Leuchter A F et al., Current psychiatry reports 12.6 (2010): 553-562). Specifically, changes in brain state that occur early in the course of treatment represent a class of dynamic biomarkers that are argued to offer especially useful prediction of outcome (Leuchter A F et al., Dialogues in clinical neuroscience 16.4 (2014): 525). Among such intermediate phenotypes, change in the resting-state electroencephalogram (EEG), and in particular change in the theta cordance biomarker (hereinafter ‘cordance’), has been linked to later clinical outcomes across a variety of interventions in MDD, including medications (Leuchter A F et al., Dialogues in clinical neuroscience 16.4 (2014): 525) and deep brain stimulation (Broadway J M et al., Neuropsychopharmacology 37.7 (2012): 1764-1772).

The cordance measure incorporates both absolute theta power (energy in mV̂2 in the 4-8 Hz frequency range) and relative power (percent theta energy relative to energy in the total spectrum) at individual recording sites across the scalp. Previous work introduced (Leuchter A F et al., Neuroimage 1.3 (1994): 208-219; Leuchter A F et al., Psychiatry Research: Neuroimaging 55.3 (1994): 141-152) and developed the cordance biomarker of treatment response in studies of selective serotonin reuptake inhibitor (SSRI) and serotonin-norephinephrine reuptake inhibitor (SNRI) antidepressant medications (Cook I A et al., Seminars in clinical neuropsychiatry. Vol. 6. No. 2. WB SAUNDERS COMPANY, 2001; Cook I A et al. Neuropsychopharmacology 27.1 (2002): 120-131; Cook I A et al., Journal of psychiatric research 39.5 (2005): 461-466; Cook I A et al., Psychiatry Research: Neuroimaging 174.2 (2009): 152-157; Leuchter A F et al., American Journal of Psychiatry 159.1 (2002): 122-129). Changes in cordance observed after the first week of treatment, particularly in the prefrontal and midline-and-right-frontal regions (Leuchter A F et al., Clinical EEG and Neuroscience 39.4 (2008): 175-181; Cook I A et al., Psychiatry Research: Neuroimaging 174.2 (2009): 152-157) have repeatedly and independently (e.g., Kopecek M et al., Neuroendocrinology Letters 27.6 (2006): 803-806; Bares Metal., Journal of psychiatric research 41.3 (2007): 319-325; Bares M et al., European Psychiatry 23.5 (2008): 350-355; Bares M et al., European Neuropsychopharmacology 20.7 (2010): 459-466) been demonstrated to predict clinical outcomes across medication classes with different mechanisms of action (MOAs) (Horacek J et al., Psychological medicine 40.09 (2010): 1443-1451). One study thus far has examined change in cordance as a predictor of response to either 1 Hz rTMS of right dorsolateral prefrontal cortex (DLPFC), or antidepressant treatment with the SNRI venlafaxine, and demonstrated equivalent capability of the prefrontal cordance biomarker to predict outcome to either intervention (Bares M et al., Clinical EEG and neuroscience 46.2 (2015): 73-80).

The present study examined change in the theta cordance biomarker obtained from whole-head EEG recordings after the first week of non-blinded flexible “dose” rTMS in a clinical research setting and sought to identify a biomarker of acute outcome. Based upon prior literature, regional changes in cordance observed over the first week of rTMS were expected to be associated with symptom improvement at the end of a six-week course of measurement-driven clinical rTMS treatment for MDD.

The materials and methods are now described.

Design

The cordance biomarker was investigated as a potential predictor of response to 6-weeks of open-label flexible-dose rTMS treatment for MDD administered in an outpatient clinical setting. qEEG recordings were obtained at pretreatment baseline and after the first week of rTMS. Changes in clinical symptoms were monitored using patient- and clinician-rated assessment scales.

Subjects

Subjects had a primary diagnosis of MDD, completed a pair of EEG recordings (baseline and week 1); and completed six weeks of open-label measurement-based care with rTMS. Subjects were clinically stable with no acute inpatient hospitalizations for at least 3 months prior to study. The majority of subjects (78%) entered rTMS treatment while continuing on one or more antidepressant medications as prescribed by their treating physicians. Medications included SSRI's (44%), SNRIs (17%), TCAs (17%), MAOIs (6%), and atypical antidepressants (28%). All subjects provided informed consent, and all procedures were carried out in accordance with the Declaration of Helsinki.

rTMS Treatment

Subjects received an acute 6-week course of TAU using the NeuroStar TMS System. Treatment sessions were scheduled for 5 weekdays per week. Treatment commenced using parameters of 3000, 10 Hz pulses per session targeting left DLPFC (L-DLFPC) with a stimulation on-time of 4 seconds, and off-time of 26 seconds (37.5 minutes total duration). Simulation intensity was titrated up to −120% of motor threshold (MT) as tolerated. Adjustments in: stimulation frequency and targeted brain region (i.e., 10 Hz left only, or sequential bilateral: 10 Hz left plus 1 Hz right DLPFC), intensity (percent MT), and number of total pulses per session (up to 5000), were made on an ongoing basis guided by the patient response using measurement-based care via weekly Inventory of Depressive Symptomatology—Self Report (IDS-SR) scores (Rush A J et al., Psychological medicine 26.03 (1996): 477-486) and physician clinical judgment, within established treatment guidelines. In general, bilateral treatment was introduced only after two weeks if patients failed to show significant benefit from left unilateral treatment.

Clinical Assessments

Changes in symptom severity were assessed at weeks 1, 2, 4, and 6 as compared to pretreatment baseline using the IDS-SR. Physician-rated symptom improvement after 6 weeks was assessed using the 7-point Clinical Global Impressions-Improvement (CGI-I) scale (Busner J et al., Psychiatry (Edgmont) 4.7 (2007): 28-37) rated by one of the treating psychiatrists. CG-I score was examined as a dichotomous outcome measurement of improvement (i.e., scores of ‘1’ very much improved, ‘2’ much improved, or ‘3’ minimally improved), versus nonimprovement (i.e., scores of ‘4’ no change, ‘5’ minimally worse, ‘6’ much worse, or ‘7’ very much worse), at the end of six weeks of treatment. The Patient Health Questionnaire (PHQ-9) was obtained prior to beginning rTMS to use as a measure of baseline illness severity independent of the IDS-SR outcome measure.

qEEG Recording and Preprocessing

Resting-state EEGs were obtained at pretreatment baseline and after the first week (5 sessions) of rTMS. The week 1 EEG was recorded just prior to beginning the second week of treatment (and not immediately following a treatment to prevent capturing any potential acute effects of stimulation).

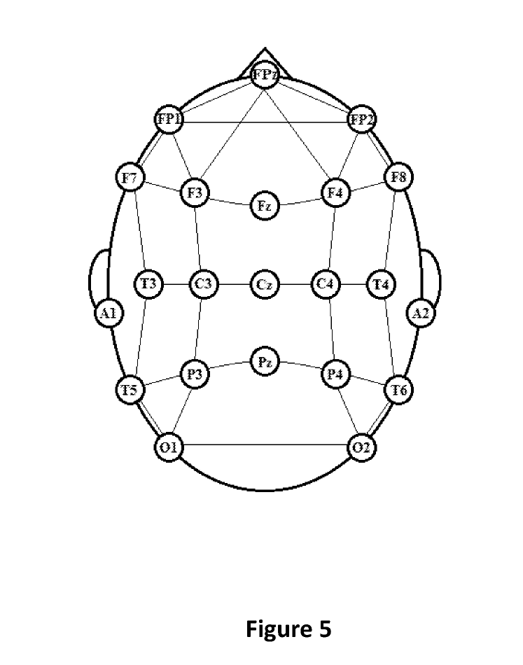

EEGs were recorded while subjects sat quietly with eyes closed with frequent alerting to avoid drowsiness, and instructions to remain still and inhibit blinks or eye movements. A customized Quasar DSI-24 recording system was employed (Wearable Sensing, Inc.; San Diego, Calif.) with a 21-channel dry electrode montage including Fpz, a Pz reference, and A2 (left Ear) ground (FIG. 5). A minimum of 10 minutes of EEG data were recorded at 16-bit resolution and a sampling rate of 300 Hz, with a low-pass filter of 70 Hz, high-pass filter of 0.5 Hz, and a notch filter at 60 Hz.

Data were stored in digital format and imported into Brain Vision Analyzer (BVA) software (Brain Products GmbH; Gilching, Germany) to remove offsets, optimize scaling, and segment into 2-s non-overlapping epochs. Any epochs containing artifacts or amplifier drift were removed using a semi-automated interactive process using the BVA artifact rejection module that utilized standard thresholds likely to represent artifact based upon voltage step gradient (i.e., change greater than 100 μV between adjacent data points), absolute values of difference within the epoch, and persistent low activity. Data was inspected using multiple bipolar and referential montages, and by consensus, data segments containing eye movement, muscle, or movement-related artifacts were removed according to standard criteria. Finally, selected data were processed using a wavelet approach to remove any cardioballistic artifact.

qEEG Variables

Cordance values were calculated from conventional qEEG absolute and relative theta power measures for each electrode site as follows. First, EEG power values were computed using a re-attributional electrode montage because this montage affords a higher correlation between EEG and cerebral perfusion than other montages (Cook I A et al., Electroencephalography and clinical neurophysiology 107.6 (1998): 408-414). Second, absolute and relative power values were z-transformed for each electrode site (s) for a given recording, yielding A_{norm(S)} and R_{norm(S)}, respectively. Third, z-score values were summed to yield a cordance “intensity” value, Z, for each electrode where Z_{(S)}=A_{norm(S)}+R_{norm(S)}. Analyses were performed using cordance measures in the theta frequency band (4-8 Hz) because previous work from this and other laboratories has indicated that energy in the theta band is most strongly associated with the effects of antidepressant treatments (Cook I A et al., Seminars in clinical neuropsychiatry. Vol. 6. No. 2. WB SAUNDERS COMPANY, 2001; Cook I A et al., Neuropsychopharmacology 27.1 (2002): 120-131; Ulrich G et al., European Archives of Psychiatry and Clinical Neuroscience 237.5 (1988): 258-263).

Change in cordance was calculated by subtracting baseline from week 1 measures for each electrode. For purposes of illustration, ‘cordance change’ values were displayed on brain topographic maps showing mean changes at each electrode for clinical improver versus non-improver groups. Cordance values were averaged across electrodes to form regional measures. Analyses examined cordance changes overlying the following seven regions: prefrontal (PFC), electrodes FP1, FPz, FP2 (cf. Cook I A et al., Neuropsychopharmacology 27.1 (2002): 120-131); midline-and-right-frontal (MRFC), electrodes Fpz, Fz, Fp2, F4, F8 (cf. Cook I A et al., Psychiatry Research: Neuroimaging 174.2 (2009): 152-157); midline-and-left-frontal (MLFC), electrodes Fpz, Fz, Fp1, F3, F7; central (CC), electrodes Fz, C3, Pz, C4, Cz; orbital (OC), electrodes O1, P3, Pz, P4, O2; midline-and-right-orbital (MROC), electrodes T6, P4, O2, Pz; midline-and-left-orbital (MLOC), electrodes T5, P3, O1, Pz. See FIG. 5 for electrode montage.

Analyses

Co-primary outcome measures were percent change in IDS-SR score and CGI-I improvement, at week 6. Regression models were used to assess relationships between week 1 changes in theta cordance and clinical outcomes after 6 weeks. Linear and logistic regression models (for continuous and dichotomous outcomes, respectively) were used to examine the predictive capability of the cordance biomarker alongside clinical and demographic covariates. Receiver operating characteristic (ROC) analysis was used to assess area under the curve (AUC) and calculate sensitivity and specificity of the cordance biomarker for predicting improver versus non-improver outcomes. Cordance biomarkers were examined in relationship to percent change in IDS-SR score at weeks 2 and 4 using Pearson's bivariate correlation.

The results are now described.

Clinical and Demographic Outcomes

Data were obtained for 18 MDD subjects (7 females, 11 males) with a mean age of 47.1 (±18.3) years and a mean baseline IDS-SR score of 40.17±10.14. Subjects had a mean baseline PHQ-9 score of 16.94±5.41. Overall, subjects reported 17.4%±0.2 improvement on the IDS-SR after six weeks of rTMS. On the CGI-I, 12 of 18 subjects (66.7%) were rated as improvers. Change in IDS-SR was not significantly associated with age, gender, or baseline PHQ-9 score (all p-values >0.23). CGI-I improvement was not significantly associated with age or gender (all p-values >0.75) but was associated with (higher) PHQ-9 (p=0.041). Early symptom change in IDS-SR score at one week was not a significant predictor of CGI-I improver status (p=0.99, N.S.)

Regional Cordance Change Predictors of Outcome

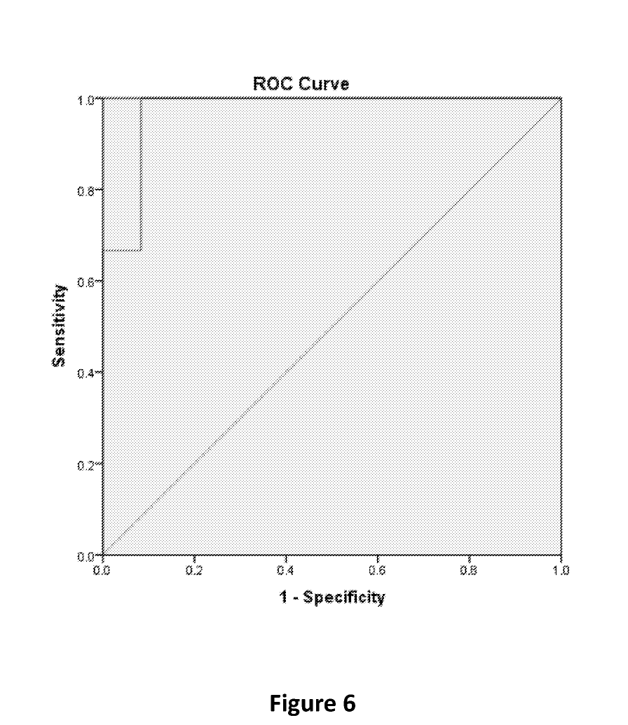

The overall model of central cordance change (CC) as a predictor of CGI-I improver status at week 6 was significant at p<0.0001 with Nagelkerke R²=0.78. This result exceeds the Bonferroni-corrected criterion for testing in seven regional groupings (i.e., 0.05/7; p≤0.007.) The CC beta coefficient (β)=−6.578 (SE=3.335) was significant (p=0.049) and indicated that for every 1 unit increase in CC, a 6.578 decrease is expected in the log odds of improvement. CC remained significant in a multivariate model that simultaneously examined age, gender, and baseline severity (PHQ-9 score) as potential predictors. Age and gender were not significant (p-values >0.73); however, greater baseline severity was associated with greater likelihood of CGI-I improvement. In a model that examined only CC and baseline severity predictors, both were significant (p=0.002, and p=0.039, respectively) with the overall model significant at p<0.0001, and Nagelkerke R²=0.890. ROC analysis of the CC predictor yielded 0.97 area under the curve (AUC) with 1.00 sensitivity and 0.92 specificity using an optimized cutpoint (FIG. 6).

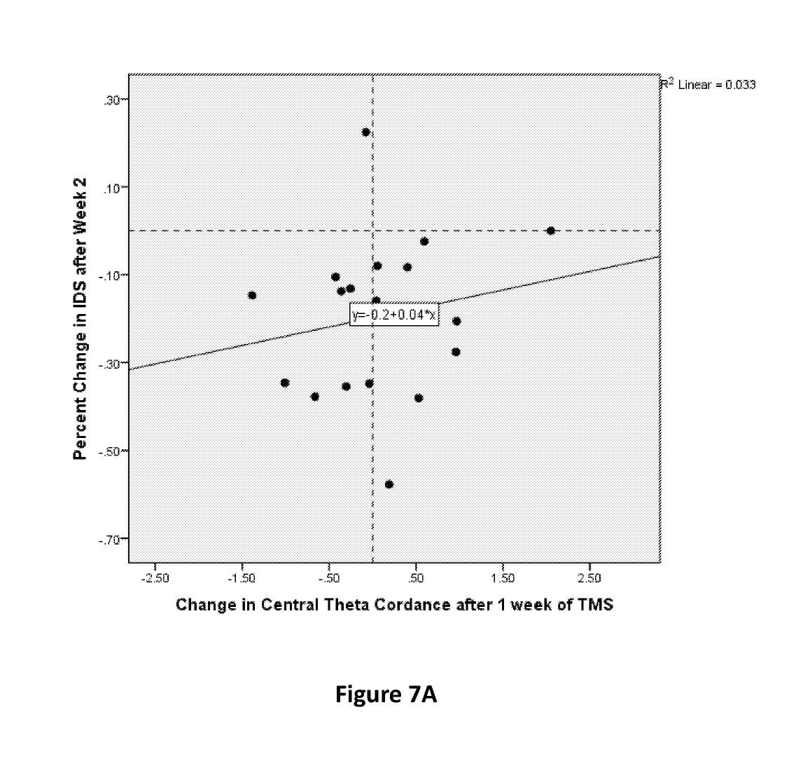

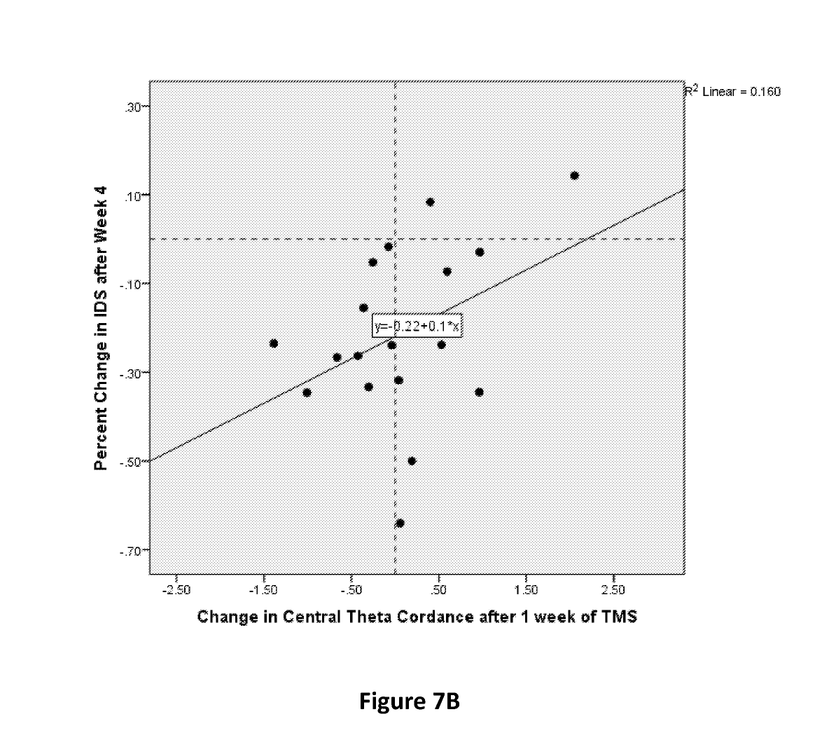

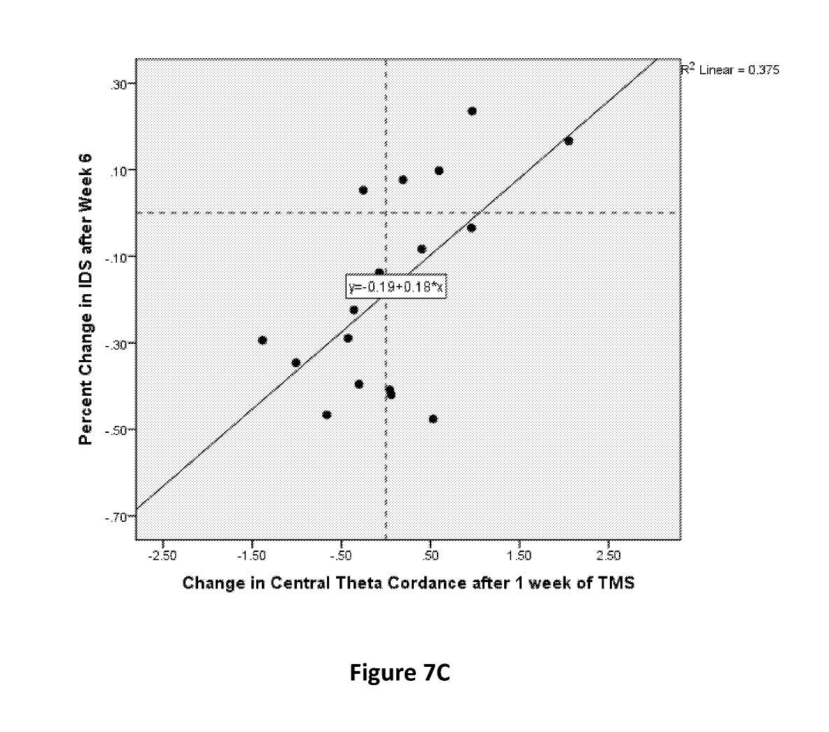

CC significantly predicted percent change in IDS-SR score at week 6 with R²=0.375, p=0.007. Baseline theta cordance was not a significant predictor (p=0.093, N.S.), and neither was percent change in IDS score at week 1 (p=0.55). The model remained significant controlling for age, gender, and baseline severity (PHQ-9 score), none of which were significant. As shown in FIG. 7A through FIG. 7C, decrease in CC was significantly associated with greater improvement at week 6 (r=0.617, p=0.007), but not at weeks 2 (r=0.181, p=0.473) or 4 (r=0.400, p=0.100).

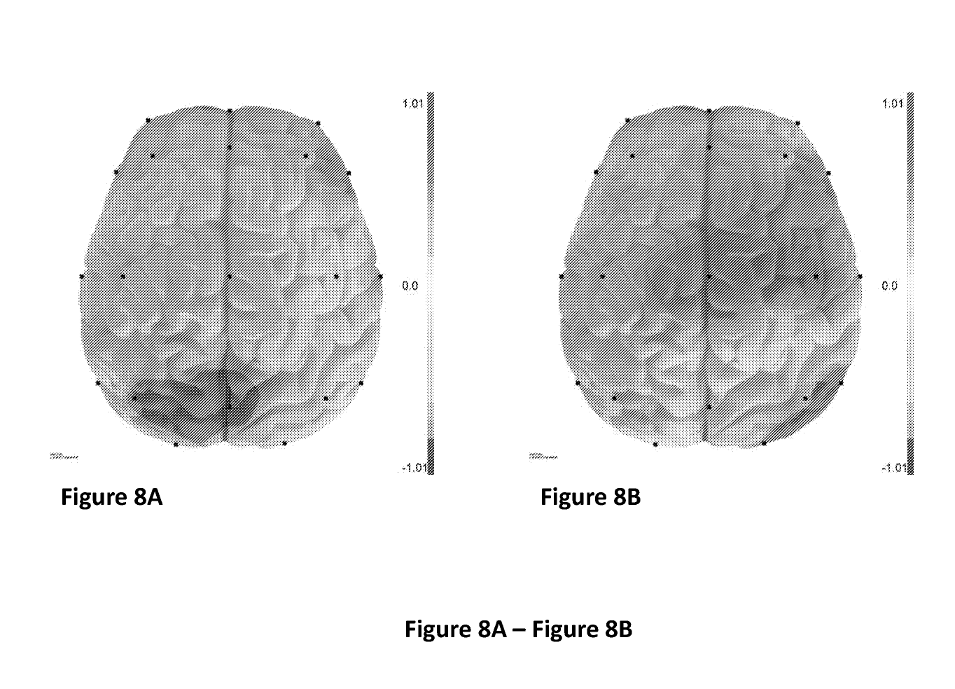

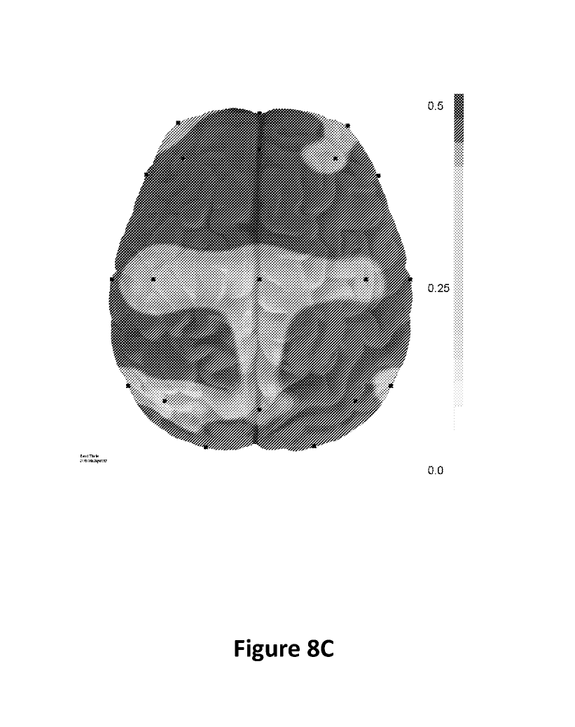

Regression analyses examining other ROI's did not attain significance. Prefrontal (PFC) and midline-and-right-frontal (MRFC) regional cordance measures did not significantly predict CGI-I (p=0.088, N.S.; p=0.168, N.S.) or IDS-SR (p=0.156, N.S.; p=0.317, N.S.) outcomes. Note also that a model using baseline theta cordane was not significant (p=0.10, N.S.) FIG. 8A shows week 1, treatment-emergent brain regional changes in theta cordance for patients classified as improvers, as compared to non-improvers in FIG. 8B. FIG. 8C identifies the locations of recording sites that showed the most highly significant differences between improvers and non-improvers.

The results of this study provide strong evidence demonstrating that the theta cordance biomarker may have utility as a predictor of outcome in the context of clinical rTMS treatment for MDD. Change in cordance overlying the central brain region (CC) after the first week of rTMS predicted both physician-rated improver status, and patient-rated magnitude of symptom improvement, at the end of six weeks of treatment as usual. Decrease in CC at week 1 was associated with later clinical improvement. CC showed greater predictive capability than clinical or demographic characteristics including age, gender, and baseline symptom severity.

Cordance changes overlying the prefrontal region (PFC) or midline-and-right frontal region (MFRC) did not significantly predict clinical outcome in this sample. PFC did show a trend relationship to CGI-I outcome (p=0.088); however, the direction of the relationship was opposite than previously observed in rTMS (Bares M et al., Clinical EEG and neuroscience 46.2 (2015): 73-80). Improvers in the study showed a numeric (non-significant) increase in PFC and in the overlapping MRFC region at week 1, whereas the study by Bares and colleagues found significant decreases in PFC at week 1 among treatment responders (Bares M et al., Clinical EEG and neuroscience 46.2 (2015): 73-80). Notably, these studies used different clinical treatment protocols: 10 Hz lDLPFC rTMS at the beginning of acute treatment in the present protocol, and 1 Hz rDLPFC rTMS throughout the course of acute treatment in the Bares study. Prior work examining cordance predictors of antidepressant medication response has consistently found prefrontal decreases associated with better clinical outcome (Cook I A et al., Neuropsychopharmacology 27.1 (2002): 120-131; Cook I A et al., Seminars in clinical neuropsychiatry. Vol. 6. No. 2. WB SAUNDERS COMPANY, 2001; Cook I A et al., Journal of psychiatric research 39.5 (2005): 461-466; Cook I A et al., Psychiatry Research: Neuroimaging 174.2 (2009): 152-157; Bares M et al., Journal of psychiatric research 41.3 (2007): 319-325; Bares M et al., European Psychiatry 23.5 (2008): 350-355; Bares M et al., European Neuropsychopharmacology 20.7 (2010): 459-466), similar to the Bares et al. result with slow rTMS (Bares M et al., Clinical EEG and neuroscience 46.2 (2015): 73-80). This is in contrast to the present finding among patients who received treatment beginning with fast rTMS. It is generally acknowledged that depending on rTMS pulse parameters, effects can be excitatory (e.g., following ‘high-frequency’ 5-20 Hz pulses; Pascual-Leone A et al., Science 263.5151 (1994): 1287-1289) or inhibitory (e.g., following ‘low-frequency’ 1 Hz pulses; Chen R et al., Neurology 48.5 (1997): 1398-1403) mimicking the effects of long-term potentiation and long-term depression. It is intriguing to speculate that increased cordance might predict outcome to high frequency stimulation, and decreased cordance might predict response to low frequency stimulation.

Treatment-emergent change in cordance overlying the central brain region has not previously been reported as a predictor of treatment outcome in MDD. Of note, one prior study examined baseline cordance regions in MDD patients treated with ECT and found that higher pretreatment cordance overlying a central region (electrodes FC1, FC2, Cz from a 35-channel recording montage) predicted better clinical outcome (Stubbeman W F et al., The journal of ECT 20.3 (2004): 142-144). In the present study cohort, higher central cordance (electrodes Fz, C3, Pz, C4, Cz) at pretreatment baseline was numerically but not significantly associated with clinical improvement. Importantly, examination of baseline, and change-from-baseline, central cordance biomarkers in the same regression model, showed that only the ‘change’ biomarker was a significant predictor of outcome. This finding lends supports to treatment-emergent IPs as predictors of outcome (Leuchter A F et al., Dialogues in clinical neuroscience 16.4 (2014): 525).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating neuropsychiatric disorders, comprising the steps of:
assessing pretreatment severity of neuropsychiatric disorder symptoms using at least one evaluation test or interview;
measuring pretreatment brain physiology and calculating at least one of pretreatment resting state delta and pretreatment resting state theta cordance using quantitative electroencephalography (qEEG);
administering a series of repetitive Transcranial Magnetic Stimulation (rTMS) treatments to a target brain region;
assessing the severity of neuropsychiatric disorder symptoms after a TMS treatment;
measuring brain physiology and calculating at least one of posttreatment resting state delta and posttreatment resting state theta cordance using qEEG after a TMS treatment; and
adjusting parameters for subsequent rTMS treatments to decrease at least one of posttreatment resting state delta and posttreatment resting state theta cordance below pretreatment resting state delta and pretreatment resting state theta cordance.

2. The method of claim 1, wherein the neuropsychiatric disorder is depression.

3. The method of claim 1, wherein the resting state delta cordance is measured between 0.5 and 4 Hz.

4. The method of claim 1, wherein the resting state theta cordance is measured between 4 and 8 Hz.

5. The method of claim 1, wherein the resting state theta cordance is measured in the central brain region.

6. The method of claim 1, wherein the at least one evaluation test or interview is selected from the group consisting of: patient health questionnaire (PHQ-9), mini mental status exam (MMSE), mini international neuropsychiatric interview (MINI), the Montgomery-Asberg depression rating scale (MADRS), the Hamilton depression scale (HAM-D), the inventory of depressive symptomatology (IDS), clinical global impression improvement (CGI-I), clinical global impression severity (CGI-S), quality of life instrument (QOLI), and the Columbia suicide severity scale (C-SSRS).

7. The method of claim 1, wherein qEEG further calculates alpha cordance, beta cordance, or both.

8. The method of claim 1, wherein the rTMS is administered to the left dorsolateral prefrontal cortex (left DLPFC).

9. The method of claim 1, wherein the rTMS is administered at a baseline of 3000 pulses at 10 Hz.

10. The method of claim 1, wherein the rTMS is administered in a continuous sequence alternating between stimulation and rest.

11. The method of claim 10, wherein the stimulation time is 4 seconds.

12. The method of claim 10, wherein the rest time is 26 seconds.

13. The method of claim 1, wherein the adjusted parameter is the location of the target brain location.

14. The method of claim 1, wherein the adjusted parameter is the rTMS magnetic frequency.

15. The method of claim 1, wherein the parameters are adjusted to decrease symptom severity as measured by HAM-D17 to a score equal to or below 7.

16. The method of claim 1, wherein the parameters are adjusted to decrease symptom severity as measured by MADRS to a score equal to or below 12.

17. The method of claim 1, wherein the parameters are adjusted to decrease symptom severity as measured by IDS-SR30 to a score equal to or below 12.

18. A system for treating neuropsychiatric disorders, comprising:
symptom severity evaluation software;
an EEG device;
a transcranial magnetic stimulation (TMS) device; and
a computer platform.

19. The system of claim 18, wherein the symptom severity evaluation software is capable of administering at least one of the group consisting of: patient health questionnaire (PHQ-9), mini mental status exam (MMSE), mini international neuropsychiatric interview (MINI), the Montgomery-Asberg depression rating scale (MADRS), the Hamilton depression scale (HAM-D), the inventory of depressive symptomatology (IDS), clinical global impression improvement (CGI-I), clinical global impression severity (CGI-S), quality of life instrument (QOLI), and the Columbia suicide severity scale (C-SSRS).

20. The system of claim 18, wherein the EEG device is capable of measuring and recording neural oscillations in the frequency range of 0.1 Hz to 20 Hz.

21. The system of claim 18, wherein the TMS device is programmable to control stimulation frequency, stimulation pattern, stimulation duration, repetition of stimulation, and stimulation intensity.
